(19) United States
(12) Patent Application Publication
(10) Pub. No.: US 2009/0234321 A1
(43) Pub. Date: Sep. 17, 2009

(54) VISUALIZATION OF CORONARY VEIN PROCEDURE

(76) Inventors: James Edward Shapland, Vadnais Heights, MN (US); Tuan Minh Doan, Burnsville, MN (US); Clifton A. Alferness, Port Orchard, WA (US)

Correspondence Address: MERCHANT & GOULD PC, P.O. BOX 2903, MINNEAPOLIS, MN, 55402-0903, US

(21) Appl. No.: 12/046,767

(22) Filed: Mar. 12, 2008

(51) Int. Cl. A61M 31/00; A61M 25/10

(52) U.S. Cl. 604/509; 604/96.01

(57) ABSTRACT

Visualization of a medical procedure to be performed in a coronary vessel (such as a coronary sinus) of a patient is achieved by the steps of: identifying a coronary vessel for a procedure and placing a catheter within the coronary vessel. The coronary vessel is occluded at a site proximal to a distal end of the catheter. A contrast medium is injected into the coronary vessel through the catheter distal end for visualization of the procedure within the coronary vessel. Following the procedure, the contrast medium is removed through the catheter distal end and occlusion of the coronary vessel is discontinued.

### I. BACKGROUND OF THE INVENTION

1. Field of the Invention

This invention pertains method and apparatus for visualizing procedures performed in a coronary vessel such as a coronary vein.

2. Description of the Prior Art

Angiography procedures are widely used for assessing patency of coronary arteries. These procedures may also be used for other purposes—for example, in stent placement or other procedures. In such a procedure, a contrast medium is injected into a coronary artery proximal to a suspected site of a coronary obstruction.

Procedures are also developed (or in development) for performing procedures in coronary veins such as the coronary sinus. By way of non-limiting example, such procedures include lead placement for bi-ventricular pacing and procedures such as those shown in U.S. Pat. No. 6,964,683 to Kowalsky et al. issued Nov. 15, 2005 and U.S. Pat. No. 6,966,926 to Mathis issued Nov. 22, 2005 for re-shaping a mitral valve.

As in coronary artery procedures, coronary vein procedures may include injecting of a contrast medium into the coronary vein to facilitate visualization of the procedure under fluoroscopy.

Contrast media may have significant health risks if permitted to flow systemically to the patient's organs. For example, renal dysfunction or failure may occur from such systemic delivery of a contrast media. Such failure is referred to as “contrast-induced nephropathy” or CIN. Schräder, “Contrast Media-Induced Renal Failure: And Overview”, Journal of Interventional Cardiology, Vol. 18, No. 6, pages 417-423 (2005).

A number of different techniques in catheter designs have been suggested for collecting contrast media. Examples of such are shown in U.S. Pat. No. 6,554,819 to Reich issued Apr. 29, 2003; U.S. Patent Application Publication No. US 2002/0099254 A1 to Movahed published Jul. 25, 2002; U.S. Patent Application Publication No. US 2005/0256441 A1 to Lotan et al., published Nov. 17, 2005, U.S. Patent Application Publication No. 2005/0124969 to Fitzgerald et al. published Jun. 9, 2005 and U.S. Patent Application Publication No. US 2006/0013772 A1 to LeWinter et al., published Jan. 19, 2006. A contrast removal system is also described in Michishita, et al. “A Novel Contrast Removal System From The Coronary Sinus Using An Absorbing Column During Coronary Angiography In A Porcine Model”, Journal of the American College of Cardiology, Vol. 47, No. 9 (2006). Collection catheters are well known for collecting blood or other fluids from blood vessels of a patient. For example, International Publication No. WO 2005/082440 A1 describes collection catheters for collecting blood from a coronary sinus or other coronary vein in a perfusion system.

### II. SUMMARY OF THE INVENTION

According to a preferred embodiment of the present invention, a method and apparatus are disclosed for visualization of a medical procedure to be performed in a coronary vessel (such as a coronary sinus) of a patient. The method includes the steps of: identifying a coronary vessel for a procedure and placing a catheter within the coronary vessel. The coronary vessel is occluded at a site proximal to a distal end of the catheter. A contrast medium is injected into the coronary vessel through the catheter distal end for visualization of the procedure within the coronary vessel. Following the procedure, the contrast medium is removed through the catheter distal end and occlusion of the coronary vessel is discontinued.

### III. BRIEF DESCRIPTION OF THE DRAWINGS

FIG. 1 is a schematic side elevation view of a catheter system according to the present invention;

FIG. 2 is a view of a proximal end of the catheter of FIG. 1 showing an alternative embodiment;

FIG. 3 is a schematic side sectional view of a coronary sinus having a catheter according to the present invention placed in the coronary sinus and shown ejecting a contrast media into the coronary sinus;

FIG. 4 is the view of FIG. 3 showing placement of a procedural tool within the coronary sinus following injection of the contrast media;

FIG. 5 is the view of FIG. 3 showing withdrawal of the contrast media from the coronary sinus;

FIG. 6 is a cross-sectional view of a first embodiment of the catheter of the present invention;

FIG. 7 is a cross-sectional view of a second cross-sectional embodiment;

FIG. 8 is a cross-sectional view of a third cross-sectional embodiment;

FIG. 9 is a longitudinal sectional view of the catheter of FIG. 6;

FIG. 10 is a longitudinal sectional view of the catheter of FIG. 8;

FIG. 11 is a longitudinal sectional view of the catheter of FIG. 7;

FIG. 12 is a end view of the catheter of FIG. 9 showing a first alternative balloon embodiment shown in an inflated state;

FIG. 13 is the view of FIG. 12 with the balloon shown in a deflated state;

FIG. 14 is the view of FIG. 12 showing an alternative embodiment with multiple balloons shown in an inflated state;

FIG. 15 is the view of FIG. 14 with balloons shown in deflated states;

FIG. 16 is a schematic side elevation view of an actuator system for the present invention;

FIG. 17 is an actuator for coordinated administration and removal of inflation fluid and contrast media; and

FIG. 18 is a graph illustrating sequence of action of the actuator of FIG. 17.

### IV. DESCRIPTION OF THE PREFERRED EMBODIMENT

Referring now to the several drawing figures in which identical elements are numbered identically throughout, a description of a preferred embodiment of the present invention will now be provided.

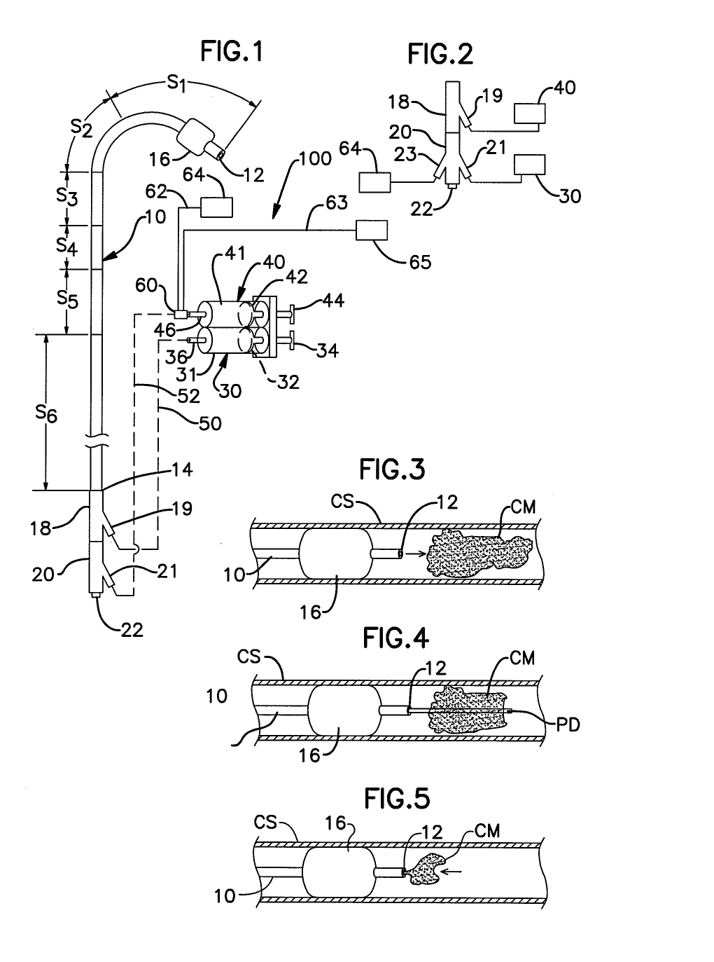

FIG. 1 shows a system 100 including a catheter 10 according to the present invention. In the preferred embodiment, the catheter 10 is used to inject a contrast media into a coronary sinus or other coronary vein. Following such injection, the catheter may be used for advancing medical procedure tools through a lumen of the catheter 10. Such procedures may include lead placement for bi-ventricular pacing or measuring devices for mitral valve re-shaping, by way of non-exhaustive examples. Following such procedure, the catheter 10 is used to remove the injected contrast media from the coronary sinus.

The catheter 10 is a long flexible and hollow tubular member 10 which has an opening at the distal end 12 and proximal end 14. The catheter 10 may be sized for delivery through a jugular vein of the patient or through a femoral access both of which are known in the art for accessing the coronary sinus. FIG. 1 shows a preferred embodiment for a jugular access catheter 10.

In a preferred embodiment a femoral access, catheter 10 will have a plurality of segments along its length of varying stiffness to facilitate advancement of the distal tip 12 into a coronary sinus of a patient. By way of non-limiting example, a first distal segment S₁ has an approximate length of about 75 mm and is relatively soft with a durometer of 35 D. The adjacent second segment S₂ has a representative length of about 50 mm and a hardness of 40 D. The next adjacent segment S₃ has a length of approximately 50 mm and a higher durometer of 55 D. The next adjacent segment S₄ has a length of approximately 50 mm and a decreased durometer of 40 D. The next adjacent segment S₅ has a length of approximately 50 mm and an increased durometer of 55 D. The most proximal length S₆ has a length of approximately 25 cm and a durometer of 63 D to 72 D. By way of non-limiting example, the catheter 10 can have a diameter of about 11 French (approximately 3.6 mm in diameter).

The proximal end 14 of the catheter 10 connects to two couplings 18 and 20, in series. First coupling 18 has a side port 19 for connection to a source of gas (e.g., air, nitrogen or helium) or saline other inflation fluid as will be described. Second coupling 20 has a side port 21 for connection to a source of a contrast media as will be described. Each of the couplings 18 and 20 has central lumens communicating with the central lumen of the catheter 10 and exposed through a most proximal port 22.

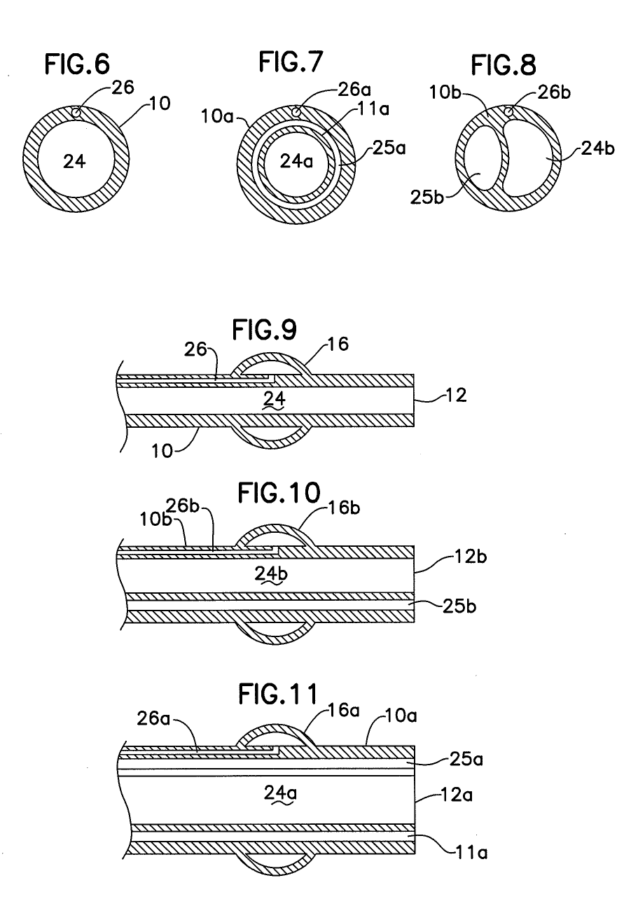

With reference to FIGS. 6 and 9, the catheter 10 is shown in a first embodiment. The catheter 10 has a central lumen 24 which is exposed through the distal tip 12. The lumen 24 is also exposed through the couplings 18 and 20 and through port 22 of FIG. 1. An inflation lumen 26 is formed in the side wall of the catheter and extends from the side port 19 of FIG. 1 to communicate with the interior of the balloon 16 as shown in FIG. 9.

Referring back to FIG. 1, the system 100 includes at least first and second fluid administrators 30, 40. In the preferred embodiment, the fluid administrators 30, 40 are shown as syringes including cylindrical bodies 31, 41 for retaining a fluid and plungers 32, 42 actuated by external members 34, 44 for movement of the plungers 32, 42 toward discharge ports 36, 46. In a most preferred embodiment, the syringes 30, 40 are fixed to one another in side-by-side relation as shown in FIG. 1 for ease of use. In all figures, syringes 30, 40 are not to scale. For example, only a few cubic centimeters (e.g., 6 cm) of gas may be need to inflate a balloon. Several hundred cubic centimeters of contrast media may be used.

Discharge port 36 is connected via tubing 50 to side port 19 of coupling 18. Discharge port 46 is connected via tubing 52 to side port 21 of coupling 20. The discharge port 46 includes an optional valve 60. In a first position, valve 60 establishes a fluid flow path between port 46 and tubing 52. In a second position, valve 60 establishes a fluid flow path between port 46 and tubing 62. In a third position, valve 60 establishes a fluid flow path between port 46 and tubing 63 connected to a reservoir 65 of fresh contrast media.

In the preferred embodiment, syringe 30 contains a fluid for inflation of balloon 16. In a most preferred embodiment, the inflation fluid is a saline or gas. At the start of a procedure, valve 60 is in the third position such that fresh contrast from reservoir 65 is drawn into syringe 40, in which syringe 40 contains a contrast medium.

In use, the catheter is advanced through the patient's vasculature until the distal end 12 resides within a coronary sinus as illustrated in FIG. 3. Plunger 32 is then depressed causing the inflation fluid to be urged through inflation lumen 26 into the balloon 16 causing inflation of the balloon 16. Inflation of the balloon 16 results in opposing surface of the balloon 16 abutting and sealing against the coronary sinus CS thereby occluding the coronary sinus CS. Following such occlusion, valve 60 is moved to the first position and plunger 40 is depressed to inject a contrast medium CM into the coronary sinus as illustrated in FIG. 3. With the contrast medium CM in the coronary sinus CS, the area of interest of the coronary sinus CS can be visualized under fluoroscopy.

With the contrast medium CM within the coronary sinus CS, procedure tools (such as lead delivery devices or measuring devices) may be advanced through the central lumen 24 by passing the procedural device PD through the proximal port 22, through the central lumen 24 and then through the distal end 12 as illustrated in FIG. 4. The procedural device PD may be left in place or removed. In either event, the contrast medium CM may be removed from the coronary sinus CS by applying a suction to the central lumen 24 causing the contrast medium CM to flow into the catheter 10 (FIG. 5). The suction is applied by drawing the plunger 42 away from port 46 so that the contrast medium is drawn through line 52. In one embodiment, valve 60 is manipulated such that a suction is applied to port 46 and fluid (e.g., contrast with blood) drawn toward syringe 40 along tubing 52 is redirected through tubing 62 to a fluid reservoir 64.

Since the recovered contrast medium CM may also contain biological hazards such as blood, the valve 60 can be shifted to the second position to move the used contrast medium from syringe 40 to reservoir 64 for subsequent disposal (or later use in the procedure if so desired). Following withdrawal of the contrast medium CM, the plunger 32 may be withdrawn thereby deflating the balloon 16 permitting removal of the catheter 10 from the patient.

Commonly, the contrast medium may be removed from the coronary sinus following visualization (venogram) and before insertion of the procedural device PD. Throughout the procedure, contrast medium can be injected or removed as desired. At the physician's option, the subsequently injected contrast medium may be fresh contrast from reservoir 65.

As mentioned, the procedural device PD may be left in place in which case the catheter 10 must be withdrawn over the procedural device PD. In a most preferred embodiment, the catheter 10 is constructed to be split along its length to allow it to be withdrawn over a procedure device PD.

It will be appreciated that such catheters capable of splitting are known in the art and an example of such is the RAPIDO® Cut-Away® Guiding Catheters of Guidant Corporation, St. Paul, Minn. USA and described in their instructions for use PPL2043029 (Mar. 10, 2004). Such catheters have hubs which may be split open along a weak point formed in the hub. A blade is then used to split the catheter along its axial length.

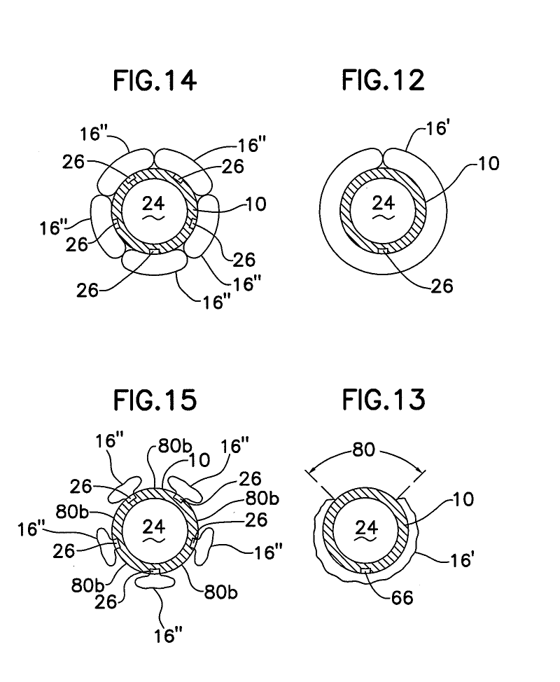

Such prior art splitting catheters do not include balloons which can otherwise obstruct the splitting process. FIGS. 12-15 show alternative designs for occlusion devices which facilitate the use of a splitting blade along the length of the catheter.

In FIGS. 12 and 13, a single balloon 16′ is shown surrounding the catheter 10. The balloon 16′ is fixed to the catheter so that a portion 80 of the catheter is exposed when the balloon is deflated as shown in FIG. 13. The exposed portion 80 permits passing of blade through the catheter wall and splitting the catheter in the region of the balloon 16′. The balloon 16′ is pre-formed so that when inflated (FIG. 12) the balloon 16′ completely surrounds the catheter 10. FIGS. 14-15 show an alternative embodiment including multiple balloons 16″ each with separate inflation lumens 26. Deflated of the balloons 16″ FIG. 15) reveals exposed portions 80b of the catheter 10 for passing a splitting blade. When inflated (FIG. 14), the balloons 16″ completely surround the catheter 10 to seal against the coronary sinus.

FIGS. 7, 8 and 11, 12 show alternative embodiments of the catheter 10. In the embodiment of FIGS. 7 and 11 the catheter 10a has a concentric inner tube 11a. The central lumen 24a resides within the inner tube 11a. Opposing surfaces of the inner tube 11a and outer tube 10a define an annular lumen 25a. An inflation lumen 26a resides within the wall of outer tube 10a. The inflation lumen 26a communicates with the interior of the balloon 16a and with side port 19. The central lumen 24a communicates only with port 22 and is for passing procedural tools or guide wires or the like through the lumen 24a. The annular lumen 25a is connected through side port 21 to the contrast medium source 40. FIGS. 8 and 10 show a still alternative embodiment very similar to FIG. 7 differing only in that the lumens 25b and 24b are not concentric but are otherwise as described with reference to FIGS. 7 and 11.

In the embodiments thus described, side port 19 acts to both admit and remove contrast media. FIG. 2 shows a still alternative embodiment with a second side port 23 in fluid communication with contrast the media lumens 25a or 25b of FIGS. 7 and 8. Side port 21 is also in communication with the same lumens 25a or 25b. Accordingly, unlike the embodiment of FIG. 1, a separate suction device (not shown) can be used to withdraw used contrast media which may be contaminated with blood through side port 23 to a reservoir 64.

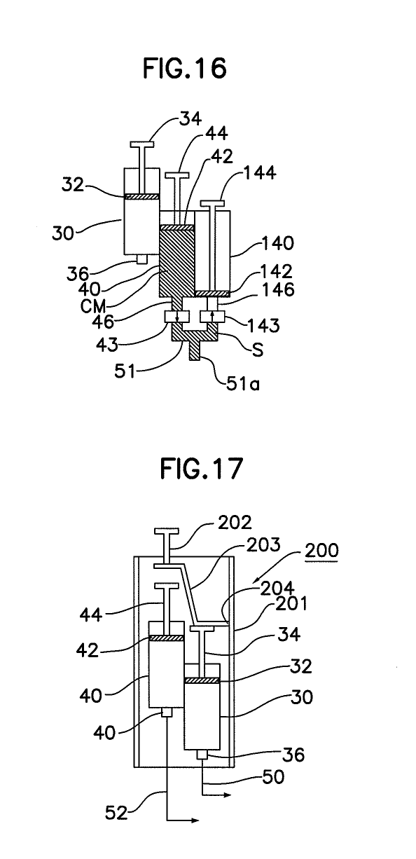

FIG. 16 illustrates a still further embodiment modifying the syringe structure shown in FIG. 1. In the embodiment of FIG. 16, syringe 40 is used only to deliver fresh contrast media through a one-way check valve 43. Fluid from the one-way check valve 43 passes into a Y-connector 51 having a port 51a connected to tubing 52 (of FIG. 1). An opposite end of the Y-connector has second syringe 140 connected to the Y-connector 51 by a one-way check valve 143. Check valve 43 permits admission of fluid from syringe 40 into the Y-connector 51. Check valve 143 permits fluid flow from the Y-connector 51 into the syringe 140. The Y-connector 51 is preloaded with saline S or the like. As shown, the syringe 140 is initially placed with the plunger 142 in a fully depressed state. With the structure thus described, syringe 40 is used to admit contrast media CM into line 52. Syringe 140 is to withdraw fluid (used contrast media mixed with blood) from line 52 into syringe 140 without such withdrawn fluid contaminating fresh contrast media contained within syringe 40.

FIG. 17 schematically illustrates a main actuator 200 for coordinated administration and removal of inflation fluid and contrast medium. The inflation cylinder 30 is connected to line 50. The contrast medium injector 40 is connected to line 52 as in FIG. 1. The administrators 30, 40 are commonly housed in a housing 201. The main actuator 200 includes a lever 203 connected to the housing 201 at a pivot point 204. The lever 203 is depressed by an exposed depression mechanism 202. The lever 203 is shaped and the administrators 30, 40 are housing in a staggered position such that upon depression of member 202, the inflation actuator 30 is first actuated followed by a delay before actuation of the contrast medium actuator 40. The reverse procedure occurs when the member 202 is withdrawn. Not shown in FIG. 17 are springs for causing plungers 34, 44, and 202 to be urged to the position shown in FIG. 17.

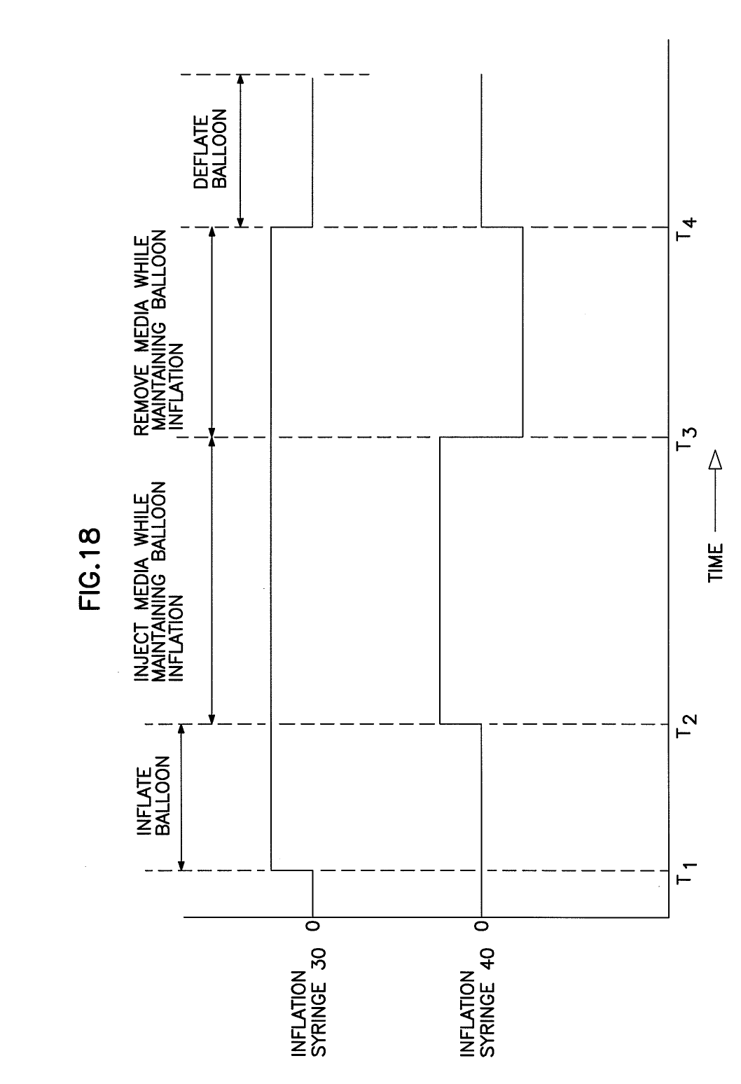

FIG. 18 shows the operation of the device of FIG. 17. The plunger 202 is depressed at times t₁. This causes plunger 34 to depress without any corresponding action by plunger 44. As a result, the balloon 16 is inflated during the stroke represented by the distance in the spacing between plunger 244 and the lever 203 at the rest position shown in FIG. 17. At time t₂, the balloon 16 is fully inflated and the lever engages plunger 44 causing injection of contrast medium while maintaining the balloon 16 in an inflated state. At time t₃, the user releases button 202 which permits the contrast medium to be withdrawn first while maintaining balloon inflation. After all the contrast medium is withdrawn at time t₄, the balloon deflates.

Having described the present invention of the preferred embodiment, modifications and equivalents will become apparent to one of ordinary skill in the art. It is intended that such modifications and equivalents be included within the scope of the claims which are appended hereto.

What is claimed is:

1. A method for visualization of a medical procedure to be performed in a coronary vessel, the method comprising:
identifying a coronary vessel for a procedure;
placing a catheter within the coronary vessel;
occluding the coronary vessel at a site proximal to a distal end of the catheter;
injecting a contrast medium into the coronary vessel through the catheter distal end for visualization of the procedure within the coronary vessel;
removing the contrast medium through the catheter distal end;
discontinuing occlusion of the coronary vessel.

2. A method according to claim 1 wherein said vessel is a coronary vein and the catheter is placed within the vein to inject the contrast medium from the catheter distal end in a direction opposing normal blood flow within the vein.

3. A method according to claim 2 wherein the coronary vein is a coronary sinus.

4. A method according to claim 1 further comprising performing the procedure by advancing tools for the procedure through the catheter and into the coronary vessel.

5. A catheter for use in visualization of a medical procedure to be performed in a coronary vessel, the catheter comprising:
a flexible, hollow, tubular catheter body having a distal end and a proximal end and with a contrast lumen extending through the catheter body and through the distal end and proximal end, said body sized to be advanced through a patient's vasculature with said distal end residing in a coronary sinus;
an occlusion member at the distal end and movable between an occlusion state and a non-occlusion state, the occlusion member occluding the coronary sinus when in the occlusion state and following insertion of the distal end in the coronary sinus;
an occlusion activation lumen formed in the catheter body and communicating with the occlusion member and accessible at a proximal end of the catheter body;
said contrast lumen communicating with actuators at the proximal end for injection and removal of contrast media from the contrast lumen.

6. A catheter according to claim 5 wherein said catheter body is split-able along an axial length of the catheter body.

7. A catheter according to claim 6 wherein the occlusion member is disconnected from a portion of the catheter body to define an unobstructed pathway for passing a splitting blade along an axial length of a distal end of the catheter body.

8. A catheter according to claim 5 wherein said occlusion member is a balloon inflatable by injection of an inflation fluid into the inflation lumen.

9. A catheter according to claim 5 further comprising a main lumen separate from the contrast lumen, the main lumen extending through the catheter body and through the distal end and proximal end.

10. A catheter system for use in visualization of a medical procedure to be performed in a coronary vessel, the catheter system comprising:
a catheter having:
a flexible, hollow, tubular catheter body having a distal end and a proximal end and with a contrast lumen extending through the catheter body and through the distal end and proximal end, said body sized to be advanced through a patient's vasculature with said distal end residing in a coronary sinus;
an occlusion member at the distal end and movable between an occlusion state and a non-occlusion state, the occlusion member occluding the coronary sinus when in the occlusion state and following insertion of the distal end in the coronary sinus;
an occlusion activation lumen formed in the catheter body and communicating with the occlusion member and accessible at a proximal end of the catheter body;
said contrast lumen communicating with actuators at the proximal end for injection and removal of contrast media from the contrast lumen;
an actuator system having:
an inflation fluid administrator releasably connected to the inflation lumen for selectively administering and removing an inflation fluid to and from, respectively, said inflation lumen;
a contrast medium administrator releasably connected to the contrast lumen for selectively administering and removing a contrast medium to and from, respectively, said contrast lumen.

11. A system according to claim 10 wherein the contrast medium administrator includes a separate fluid injector and a separate fluid remover both connected to the contrast lumen for selectively administering and removing, respectively, the contrast medium.

12. A system according to claim 10 further comprising a main actuator for coordinated actuation of the inflation fluid administrator and the contrast medium administrator
